(19) United States
(12) Patent Application Publication
(10) Pub. No.: US 2018/0055354 A1
(43) Pub. Date: Mar. 1, 2018

(54) NOVEL NON-INTRUSIVE APPROACH TO ASSESS DROWSINESS BASED ON EYE MOVEMENTS AND BLINKING

(71) Applicant: Alcohol Countermeasure Systems (International) Inc., Toronto (CA)

(72) Inventors: Ali Shahidi Zandi, Toronto (CA); Azhar Quddus, Toronto (CA); Felix J.E. Comeau, Toronto (CA); Stuart Fogel, Toronto (CA)

(21) Appl. No.: 15/692,499

(22) Filed: Aug. 31, 2017

Related U.S. Application Data
(60) Provisional application No. 62/381,631, filed on Aug. 31, 2016.

(51) Int. Cl. A61B 3/113; A61B 5/11; A61B 5/00

(52) U.S. Cl. CPC A61B 3/113; A61B 5/4082; A61B 5/1103

(57) ABSTRACT

A method, which is for assessing drowsiness of a subject over a period of time, includes the steps of: acquiring gaze and blink measurements of the subject over a period of time; and statistically comparing the measurements against a plurality of gaze and blink measurements, which have been correlated to alertness, in order to produce a value representative of alertness/drowsiness.

### CROSS-REFERENCE TO RELATED APPLICATION

This application claims the benefit of U.S. Provisional Patent Application Ser. No. 62/381,631, filed Aug. 31, 2016.

### BACKGROUND OF THE INVENTION

### 1. Field of the Invention

The present invention is directed to a non-intrusive method to assess drowsiness based on eye movements and blinking.

### 2. Prior Art

Sleep loss has reached epidemic proportions. It is estimated that 50-70 million Americans suffer from sleep disorders [1], and on average, one gets 20% less sleep than a century ago [2]. Sleep deprivation results in increased drowsiness, fatigue, and cognitive deficits, which can have a negative impact on health, safety and performance [3], and even deadly consequences. Nearly 3% of crash fatalities in 2014 involved drowsy driving on US roadways [4], with more than 80,000 sleep-related crashes each year. Accordingly, development of reliable real-time systems to identify impaired vigilance could reduce the risk of fatigue-related accidents.

### SUMMARY OF THE INVENTION

Forming one aspect of the invention is a method for assessing drowsiness of a subject over a period of time. In the method, gaze and blink measurements of the subject are acquired over a period of time and statistically compared against a plurality of gaze and blink measurements, which have been correlated to alertness, in order to produce a value representative of alertness/drowsiness. The comparison can be based upon a Guassian mixture model (GMM). The assessment of drowsiness can use reaction times to visual stimuli during a psychomotor vigilance task as the objective determinant of alertness. The gaze and blink measurements used in the drowsiness assessment can be those collected in a predetermined period immediately preceding each PVT stimulus.

### BRIEF DESCRIPTION OF THE DRAWINGS

FIG. 1(a) shows Drowsiness (raw) index and reaction (raw) time for a PVT episode from Subject 2;

FIG. 1(b) shows Drowsiness index and reaction time mapped into [−1,1] using a piece-wise-linear model; and

FIG. 2 shows the normalized RMS error between the GMM-based drowsiness index and reaction time (after mapping) for all subjects together, reported for the proposed method for both NS and SR sessions, in comparison to a random estimator.

### DETAILED DESCRIPTION OF THE PREFERRED EMBODIMENTS

The embodiments discussed herein are merely illustrative of specific manners in which to make and use the invention and are not to be interpreted as limiting the scope.

While the invention has been described with a certain degree of particularity, it is to be noted that many modifications may be made in the details of the invention's construction and the arrangement of its components without departing from the scope of this disclosure. It is understood that the invention is not limited to the embodiments set forth herein for purposes of exemplification.

Experimental

The experimental methodology is based on learning a GMM [5] of the state of alertness and measuring the distance between the observed state and the reference model. Due to the variation within the alert state, i.e. existence of sub-clusters, a GMM estimator (with a flexible number of components) would be more intuitive. In a study, the reaction times to visual stimuli during a psychomotor vigilance task (PVT) [6] were used as the baseline. The experiment included 6 episodes of 10-min PVT, each consisting of 100 stimuli-response trials. Throughout the experiment, the subject was under surveillance using an infra-red-based eye tracking system continuously acquiring gaze and blink measurements. For each PVT stimulus, a 10-second window immediately preceding that stimulus and extracted a set of 25 features (Table 1) from the corresponding eye tracking data (i.e. 600 feature vectors per experiment) was considered. Each feature vector was then considered as an observation and linked to the reaction time to the corresponding stimulus. After splitting each subject's data into separate training and test sets, the training observations representing the alertness (based on the corresponding reaction times) were used to build the GMM for each subject. Moreover, dimensionality of the feature vector was reduced to 10 by Fisher's discriminant analysis after estimating a projection matrix using the training set. Finally, given an observation, the minimum Mahalanobis distance logarithm between that observation and centres of GMM components was computed as a raw index and then mapped into [−1,1], using a piece-wise-linear model with saturation, to calculate the drowsiness index.

TABLE 1
List of the extracted features

| | | | |
|---|---|---|---|
| Gaze SD** in | Fixation | Saccade | Blinking |
| x- and y-coor- | duration | duration | duration |
| dinates | | | |
| Gaze median in | Fixation | Saccade | Blinking |
| x- and y-coor- | frequency | frequency | frequency |
| dinates | | | |
| Gaze scanpath in | Fixation time | Saccade time | Blinking time |
| x- and y-coor- | percentage | percentage | percentage |
| dinates | | | |
| Gaze velocity in | Fixation scanpath | Saccade scanpath | |
| x- and y-coor- | in x- and y-coor- | in x- and y-coor- | |
| dinates | dinates | dinates | |
| | Fixation velocity | Saccade velocity | |
| | in x- and y-coor- | in x- and y-coor- | |
| | dinates | dinates | |

**standard deviation

Eye tracking data was acquired using the GazePoint GP3 Eye Tracker from 15 participants (age 22.9±3.3 years; 11 female) at the Brain and Mind Sleep Research Laboratory, Western University, Canada. Each subject participated in two sessions with different sleep requirements: normal sleep (NS) and sleep restriction (SR) sessions, spaced at least 72 hours apart. During the night prior to NS session, the subject was required to have extended sleep for 9 hours (12-9 am), while in case of SR session, the sleep was restricted to 5 hours (1:30-6:30 am). The subject's compliance with these requirements was verified using a sleep log and actigraphy.

### Results

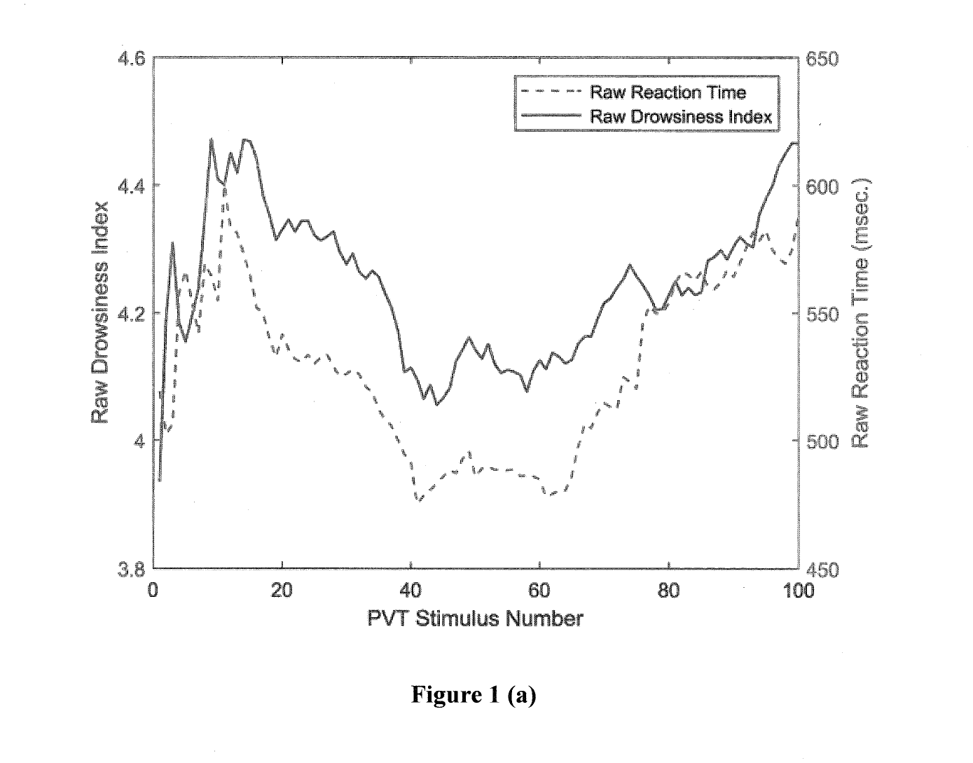

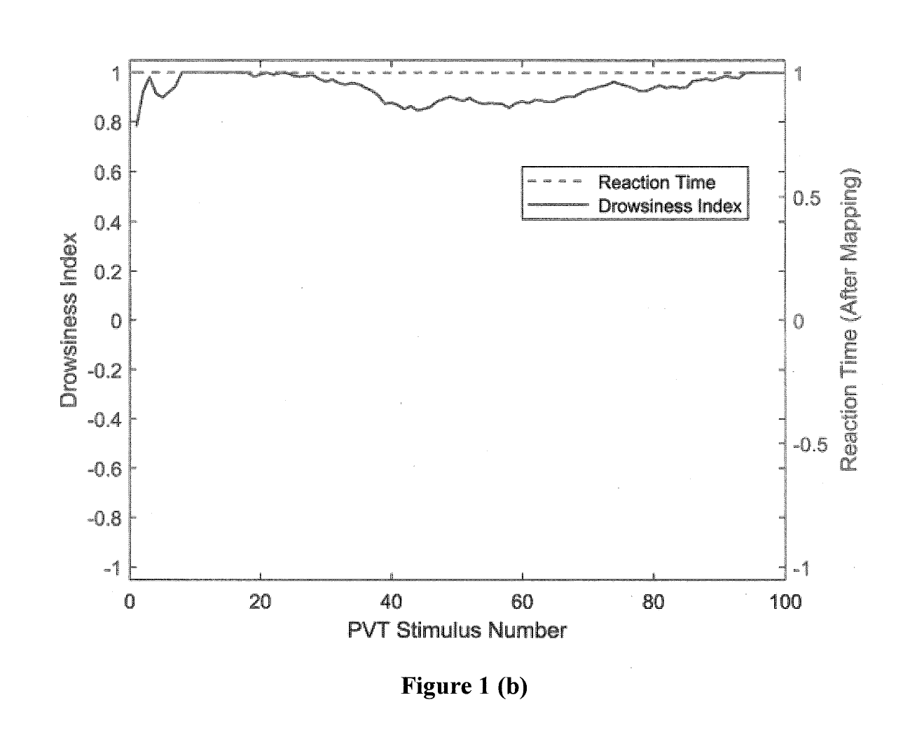

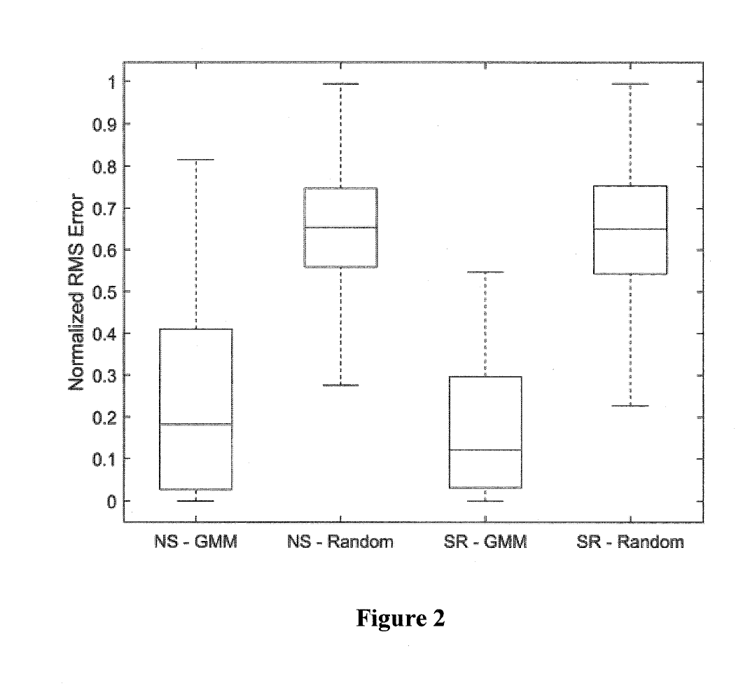

The method was evaluated on the data acquired from each subject in every session (NS or SR) using a leave-one-out cross-validation approach; i.e., choosing one PVT episode for validation each time and using the remaining episodes for training. For evaluation purpose, the corresponding reaction times were also mapped into [−1,1] using a piece-wise-linear model with saturation. The normalized root-mean-square (RMS) error between the drowsiness index and the corresponding reaction time was then calculated to assess the performance. Furthermore, the performance of the proposed method was compared to a random estimator. Overall, the proposed method shows low normalized RMS errors for both NS and SR sessions, while outperforming the random estimator (FIGS. 1-2). Taken together, these results suggest a high correspondence between features extracted from eye tracking and reaction time during a sustained vigilance task (as discussed below).

### Discussion

As an example at the individual level, FIG. 1 depicts the proposed GMM-based drowsiness index and the corresponding reaction times for a PVT episode in an SR session (Subject 2). According to the reaction time values (all greater than 475 ms), the subject can be considered drowsy for the whole episode.

As shown in FIG. 1(a), the raw drowsiness index correlates well with the reaction time (r=0.79, p<0.001), while the drowsiness index shows a small deviation (0.04 of RMS error) from the reaction time after mapping (FIG. 1(b)).

FIG. 2 shows the overall performance of the proposed GMM-based methodology for all subjects together (both NS and SR sessions) in comparison to a random estimator. As shown, the median normalized RMS error between the drowsiness index and reaction time is less than 0.2 for both sessions, suggesting high correspondence between the proposed index and the baseline. Moreover, the normalized RMS error for GMM-based method is significantly lower than the random estimator (p<0.001).

On the other hand, the RMS error for the NS session is higher than SR (p<0.05), which is expected due to the sleep deprivation effect causing stronger discrimination between the alert and drowsiness states during the SR session.

Results of this preliminary study verify the potential of the proposed methodology as a reliable approach for non-intrusive assessment of drowsiness, based on eye movements and blinking.

Since the reaction time can also be influenced by other factors such as distraction or disengagement, in future studies, other biological measures, such as electroencephalogram (EEG) and electrocardiogram (ECG), might be utilized to have a more reliable baseline.

### CONCLUSION

Several methodologies for evaluating human vigilance and fatigue have been developed in the recent past, e.g. for drivers [7]. However, major limitations of these techniques are that they may detect sleepiness too late to effectively prevent fatigue-related accidents, may not be robust under various environmental conditions, can be poorly evaluated, and/or can be intrusive.

The present invention is a non-intrusive drowsiness detection technique based which relies on features extracted from eye movements and blinking. The technique presents relatively high correspondence with reaction times. Importantly, the proposed methodology significantly outperforms a random estimator.

This invention poses the potential to lead development of non-intrusive real-time techniques to reliably assess the state of vigilance, which is useful for managing fatigue in people and reducing motor vehicle collisions and human fatalities.

Whereas, the invention has been described in relation to the drawings attached hereto, it should be understood that other and further modifications, apart from those shown or suggested herein, may be made within the scope of this invention.

### REFERENCES

[1] M. Tjepkema, “Insomnia,” Heal. Rep., vol. 17, no. 1, pp. 9-25, 2005.
[2] NCSDR (National Commission on Sleep Disorders Research), “Wake Up America: A National Sleep Alert. Volume II: Working Group Reports,” Washington, D C, 1994.
[3] S. Banks and D. Dinges, “Behavioral and Physiological Consequences of Sleep Restriction,” J. Cinical Sleep Med., vol. 3, no. 5, pp. 519-528, 2007.
[4] NHTSA (National Highway Traffic Safety Administration), “Drowsy Driving.” [Online]. Available: https://www.nhtsa.gov/risky-driving/drowsy-driving.
[5] C. M. Bishop, “Mixture Models and EM,” in Pattern Recognition and Machine Learning, Springer, 2006, pp. 423-460.
[6] S. P. a Drummond, A. Bischoff-Grethe, D. F. Dinges, L. Ayalon, S. C. Mednick, and M. J. Meloy, “The Neural Basis of the Psychomotor Vigilance Task,” Sleep, vol. 28, no. 9, pp. 1059-1068, 2005.
[7] Y. Dong, Z. Hu, K. Uchimura, and N. Murayama, “Driver Inattention Monitoring System for Intelligent Vehicles: A Review,” IEEE Trans. Intell. Transp. Syst., vol. 12, no. 2, pp. 596-614, Jun. 2011.

What is claimed is:

1. A method for assessing drowsiness of a subject over a period of time, comprising:
acquiring gaze and blink measurements of the subject over a period of time; and
statistically comparing the measurements against a plurality of gaze and blink measurements, which have been correlated to alertness, in order to produce a value representative of alertness/drowsiness.

2. A method according to claim 1, wherein the comparison is based upon a Guassian mixture model (GMM).

3. A method according to claim 1, wherein the assessment of drowsiness can use reaction times to visual stimuli during a psychomotor vigilance task as the objective determinant of alertness.

4. A method according to claim 3, wherein the gaze and blink measurements used in the drowsiness assessment can be those collected in a predetermined period immediately preceding each PVT stimulus.
